(19) United States
(12) Patent Application Publication
(10) Pub. No.: US 2020/0281733 A1
(43) Pub. Date: Sep. 10, 2020

(54) Mini Bicondylar Knee Implant and Method for Insertion Through Direct Lateral Approach and Instrumentation for Fixation with Insertable Compression Clips

(71) Applicant: JOINT INNOVATION TECHNOLOGY, LLC., Boca Raton, FL (US)

(72) Inventor: Zafer TERMANINI, Port Saint Lucie, FL (US)

(21) Appl. No.: 16/804,783

(22) Filed: Feb. 28, 2020

Related U.S. Application Data
(60) Provisional application No. 62/815,731, filed on Mar. 8, 2019.

(51) Int. Cl. A61F 2/38

(52) U.S. Cl. CPC A61F 2/3859; A61F 2002/3863; A61F 2/389

(57) ABSTRACT

A bicondylar knee prosthesis configured to be implanted through a direct lateral approach during which insertion, the femoral and tibial components are affixed and secured to the bone using shaped compression clips inserted laterally into the femoral and tibial components having correspondingly configured receiving channels.

The present invention generally relates to a knee joint implant and more specifically to a mini bicondylar knee resurfacing prosthesis adapted to be inserted through a direct lateral approach. The present invention also relates to insertable fixation means, which provide improved attachment with simultaneous compression of the implant onto surrounding bone.

Knee resurfacing implants have been in use for decades. Conventionally, the distal end of the femur, the proximal end of the tibia and the articular surface of the patella are surgically removed and replaced by the corresponding components of an implant. Bone cement was initially used to provide fixation of these implant components to bone. More recently, cementless implants gained popularity where fixation is provided by bone ingrowth into the surface asperities of the implant. Nonetheless, often the limited contact surface of the implant made it difficult to ensure stable initial implant fixation to the bone.

Implants of the prior art attempted to use septum, pegs and alike for fixation, which are fixed to the implant and thus a necessary or integral part of their design. While such frequently provided improved resistance to rotation of the implant relative to the bone, such do not provide any compression of the implant against bone.

I have previously described a mini bicondylar knee resurfacing prosthesis adapted to be inserted through a direct lateral approach in PCT patent application PCT/US2017/018058, as well as in U.S. Pat. No. 8,114,164. However, the mini bicondylar knee resurfacing prosthesis described hereafter is distinguishable over the foregoing.

Shortcomings of the prior art are addressed and overcome by various aspects of my present invention.

One aspect of my invention is a bicondylar knee resurfacing prosthesis which substantially departs from the conventional concept and designs of the prior art, and in so doing provides a novel design for providing fixation of my bicondylar knee implant to bone, particularly in view of the fact that the design of my knee implant was primarily developed for the purpose of resurfacing the articular surfaces of the femur and the tibia through a limited and true lateral approach to the knee without disruption of the extensor mechanism or damage to the quadriceps femoris tendon. Furthermore, the prosthesis of the present invention provides a novel fixation means in a form of insertable shaped retaining clips (also, sometimes referred to as ‘compression clips’) which are slidably inserted through the same lateral approach.

Furthermore, in accordance with a preferred embodiment, the shaped retaining clips of my invention are made of or comprise a resorbable material, which material is strong enough to provide the initial intended fixation and compression of a component of my bicondylar knee resurfacing prosthesis against a bone, (preferably against resurfaced bony surfaces) but which resorbable material will be absorbed within 60 to 90 days when bone ingrowth has taken place. In a particularly preferred embodiment disclosed amongst my drawing figures the shaped retaining clips have a cross-sectional geometry similar to an “I-beam” shape, that is to say having a plate shaped web part having depending from opposite longitudinal edges thereof plate shaped top and bottom sections which are preferably perpendicularly affixed to an intermediate interconnecting web part. The cross section is preferably symmetrical about the mid-point of the connector section at a cross-section thereof, although such is not a strict requirement of a shaped retaining clip.

By use of my bicondylar knee resurfacing prosthesis and shaped retaining clip(s) system, a major problem associated with conventional knee replacement implants is that the cruciate ligaments are often sacrificed during surgery. This is disadvantageous as the anterior and posterior cruciate ligaments provide stability to the knee joint during range of motion and play major role in stability of the knee during daily activity. In this respect, the bicondylar knee resurfacing prosthesis and I beam clip(s) system according to the present invention retain the anterior and posterior cruciate ligaments, and in doing so post-operatively preserve the natural knee motion and improve knee function of the patient.

A significant advantage of my invention is in preservation of the Anterior Cruciate Ligament (ACL) during the lateral insertion of the femoral and tibial components. This will preserve the physiological function of the knee and allow range of motion to be kinematically as close as possible to normal knee.

An additional advantage of the present invention is in the provision of increased stability of the implanted components of the prosthesis, particularly the reduced likelihood of (or reduced actual) sliding movement of the implanted components, thereby reducing wear of components of the prosthesis, thus increasing the longevity of my bicondylar knee resurfacing prosthesis.

My bicondylar mini knee resurfacing prosthesis wherein the implantable femoral and tibial components my bicondylar mini knee resurfacing prosthesis can be inserted through a relatively small incision via a direct lateral approach. preserves bone as the implantable components are generally of a much smaller size than comparable components of prior art knee prosthetics which are typically inserted through a direct anterior approach to the knee, and my implantable components require a very limited bone resection surface and limited bone removal. Said bone stock preservation is very beneficial both immediately as well as during any subsequent revision surgery.

Another object of the present invention is to provide novel fixation means of the implant to bone. Said fixation means are shaped retaining clips, preferably clips which have an I-beam shaped cross-section which may be slidably inserted through the same lateral approach used in implanting the femoral and tibial components, into specifically designed corresponding receiving channels which are present in one or both of the femoral and tibial components. Such receiving channels have a cross-section which is shaped to receive a part of the shaped retaining clips. Where the shaped retaining clips are of the preferred, I beam shaped cross-sectional configuration previously discussed, then correspondingly dimensioned “T” shaped receiving channels are provided within the femoral and/or tibial components of my bicondylar mini knee resurfacing prosthesis. Preferred embodiments of receiving channels and correspondingly shaped retaining clips are depicted amongst the drawing figures forming part of this application. It is to be understood however that I beam shaped retaining clips and corresponding T shaped channels are a preferred, but are a non-limiting embodiment of my invention as other complementary shapes for the shaped retaining clip and receiving channels may be produced and used to attain the same benefits as according to my preferred embodiment. For example a shaped retaining clip may have a cross-section in an “hourglass” shape or a “dumbbell” shape or a “butterfly” shape, and the receiving channels are of a corresponding cross-sectional shape to receive such a shaped retaining clip when laterally inserted as described with reference to my preferred embodiment. Still further cross-sectional geometries for both the shaped retaining clip and the receiving channels are possible and foreseen, it is only required that a narrowed connector section be present in the shaped retaining clip.

Yet further advantage of the present invention is to provide an absorbable fixation means used to provide initial compression of an implanted component against bone surface; such offers significant benefits using cementless implant since fixation is provided by bone ingrowth into the interstices and asperities of the non-articular surface of the implant. Said shaped retaining clips will become ingrown into the bone, or will be absorbed within 60-90 days.

There has been thus outlined, rather broadly, the more important feature of the invention in order that the detailed description thereof may be better understood, and in order that the present contribution to the art may be appreciated.

It is to be understood that the invention is not limited in its application to the details of construction and to the arrangement of the various parts and components used to provide my bicondylar knee resurfacing prosthesis and shaped retaining clip(s) system. The invention is capable of other embodiments and of being practiced and carried out in various ways. Furthermore, this invention may be embodied in the form illustrated in the accompanying drawings, attention being called to the fact, however, that the drawings are illustrative only and that changes may be made in the specific construction illustrated.

Features and advantages of the present invention will become fully appreciated when considered in conjunction with the accompanying drawings, in which like reference characters designate the same or similar parts throughout the several views, and wherein:

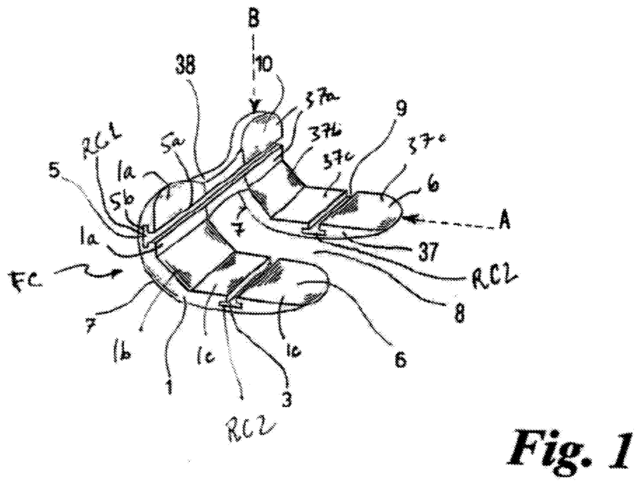

FIG. 1 is an antero lateral view of a femoral component exposing the receiving channels, here T shaped receiving channels;

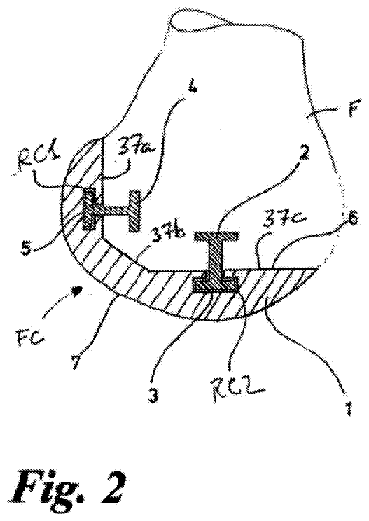

FIG. 2 is a cross sectional view of the femoral component through reference plane defined by line A and line B, illustrating its placement in interfacial contact with resected surfaces of a femur and inserted I beam shaped retaining clips;

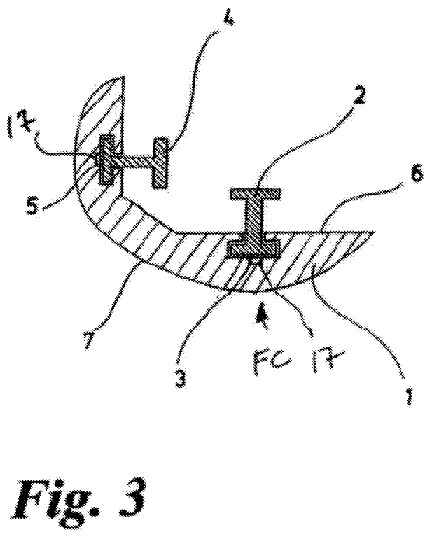

FIG. 3 is a cross sectional view of the femoral component through reference plane defined by line A and line B and inserted I beam shaped retaining clips;

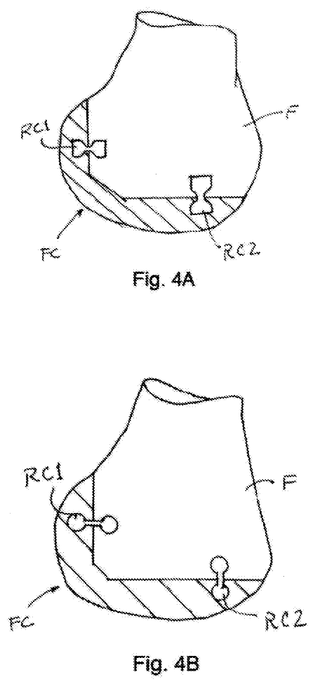

FIG. 4A illustrates a cross-sectional view of the femoral component through reference plane defined by line A and line B (c.f. FIG. 2) illustrating its placement in interfacial contact with resected surfaces of a femur and inserted hourglass shaped retaining clips.

FIG. 4B illustrates a cross-sectional view of the femoral component through reference plane defined by line A and line B (c.f FIG. 2) illustrating its placement in interfacial contact with resected surfaces of a femur and inserted dumbbell shaped retaining clips.

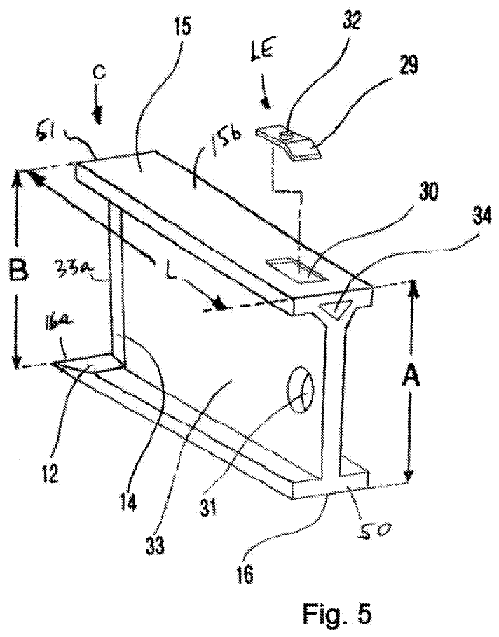

FIG. 5 is a perspective view of the shaped retaining clip;

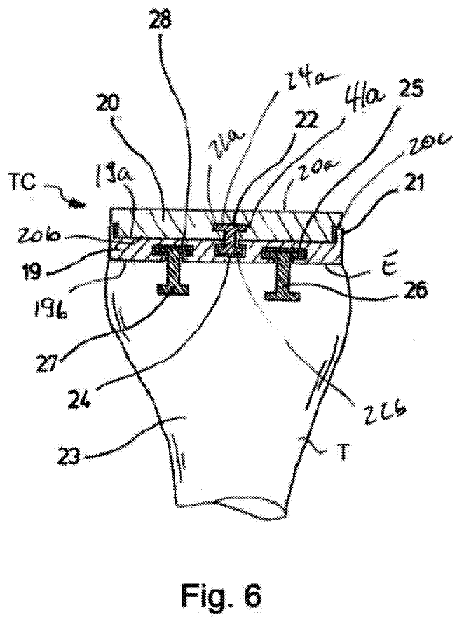

FIG. 6 is a cross sectional sagittal view centrally passing through the proximal tibia showing a tibial component and its (polyethylene) insert secured to a tibial tray with compression retaining clips and a locking lip. The tibial tray is also secured the bone with shaped retaining clips;

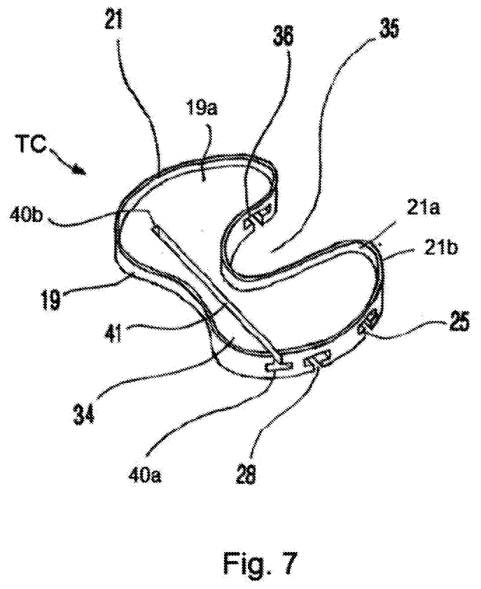

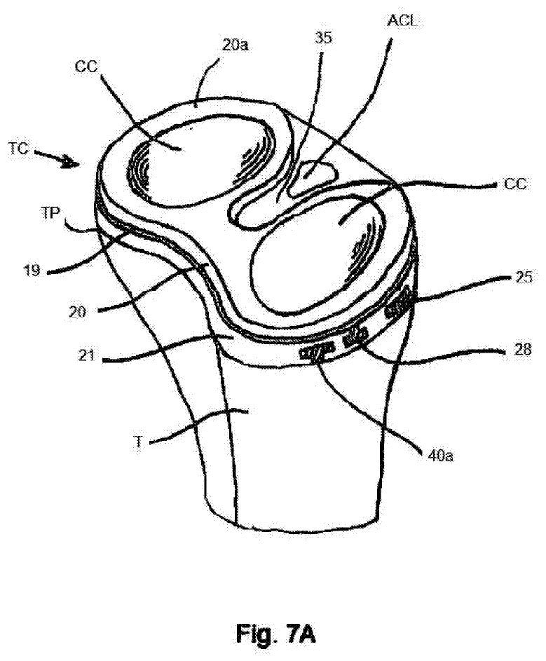

FIG. 7 is a perspective view of the bicondylar tibial tray of FIG. 6, here revealing the receiving channels, (T shaped receiving channels) configured to receive correspondingly shaped retaining clips which affix the tibial tray in interfacial contact with a resected surface of a tibia; and also seen is a T shaped channel used in retaining the polyethylene component.

FIG. 7A illustrates a perspective view of the bicondylar tibial tray of FIG. 7, including an insert, and mounted on the end of a tibia.

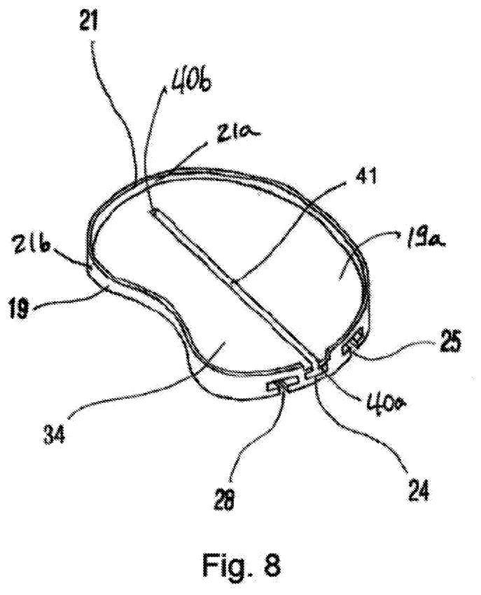

FIG. 8 is a perspective view of further embodiment of a bicondylar tibial tray, also illustrating receiving channels (T shaped receiving channels) configured to receive correspondingly shaped retaining clips which affix the tibial tray in interfacial contact with a resected surface of a tibia; also seen is a T shaped channel used in retaining the polyethylene component.

FIG. 8A illustrates a perspective view of the bicondylar tibial tray of FIG. 8, including an insert, and mounted on the end of a tibia.

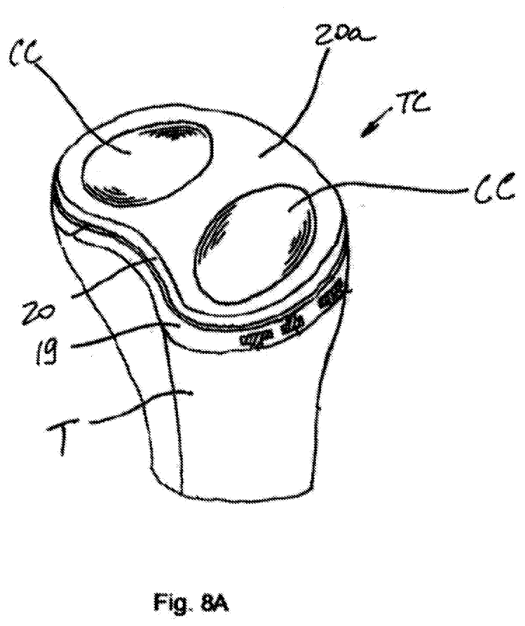

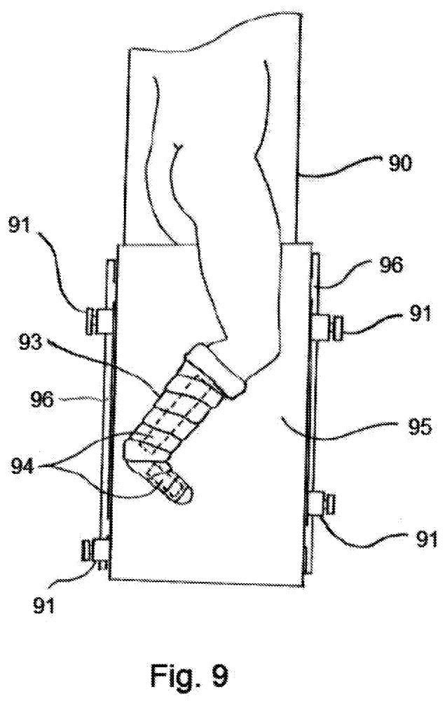

FIG. 9 depicts the placement of a patient upon an operating table, and the positioning of the knee.

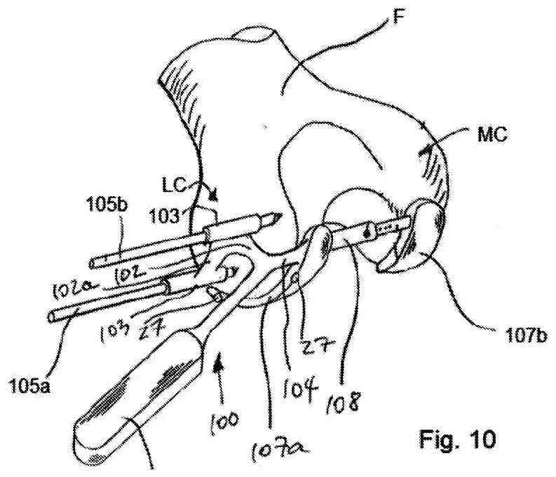

FIG. 10 illustrates a hand held femoral template with respect to a femur.

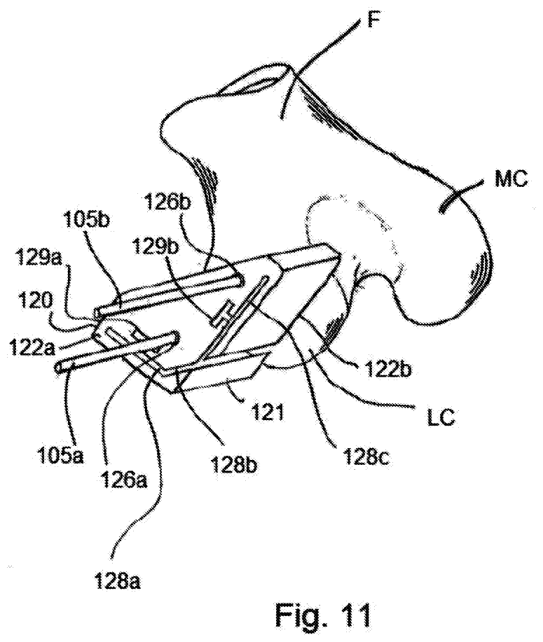

FIG. 11 depicts a cutting block used to delineate the position of resected surfaces and of retaining clips relative to resected surfaces.

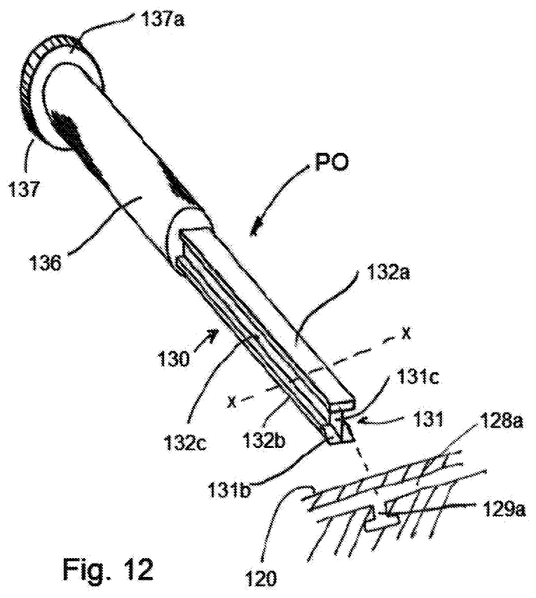

FIG. 12 illustrates in a perspective view a penetrating osteome used with the cutting block of FIG. 11.

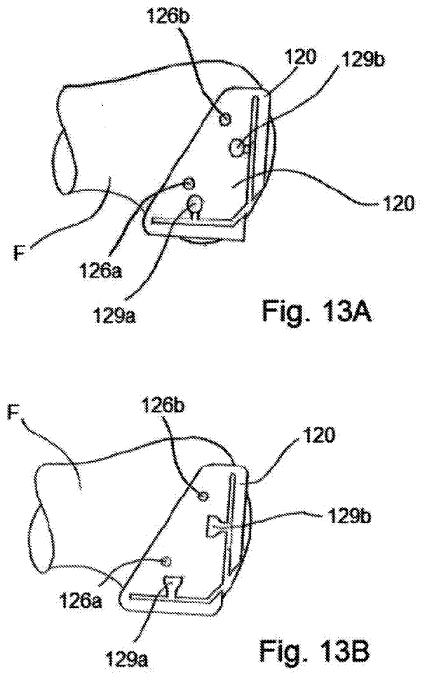

FIG. 13A depicts a first alternative embodiment of a cutting block generally in accordance with

FIG. 11.

FIG. 13B depicts a second alternative embodiment of a cutting block generally in accordance with FIG. 11.

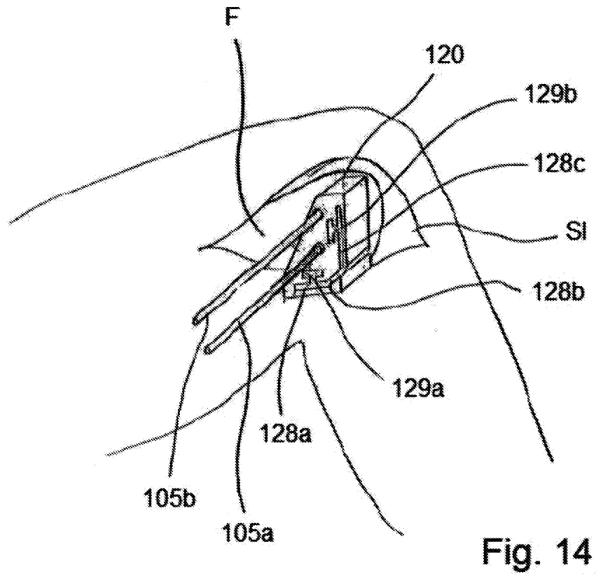

FIG. 14 illustrates a cutting block removably mounted upon stabilizer pins over the lateral aspect of the distal femur at the proximal end of the surgical incision illustrating its lateral location relative to the knee joint.

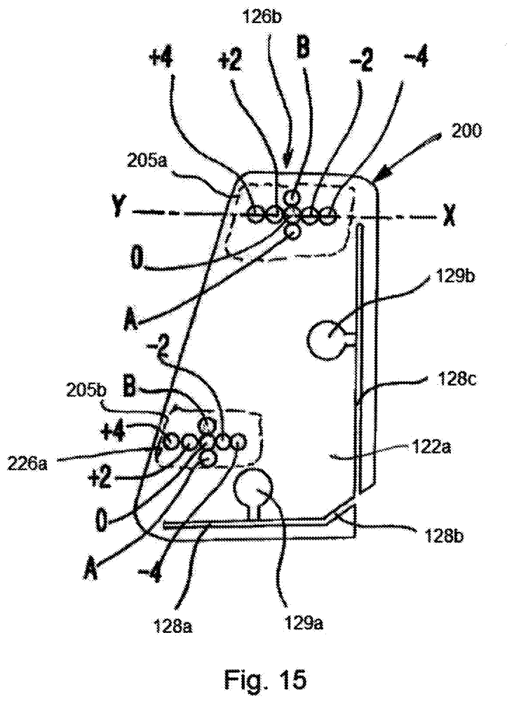

FIG. 15 depicts a side view of multi-perforate cutting block.

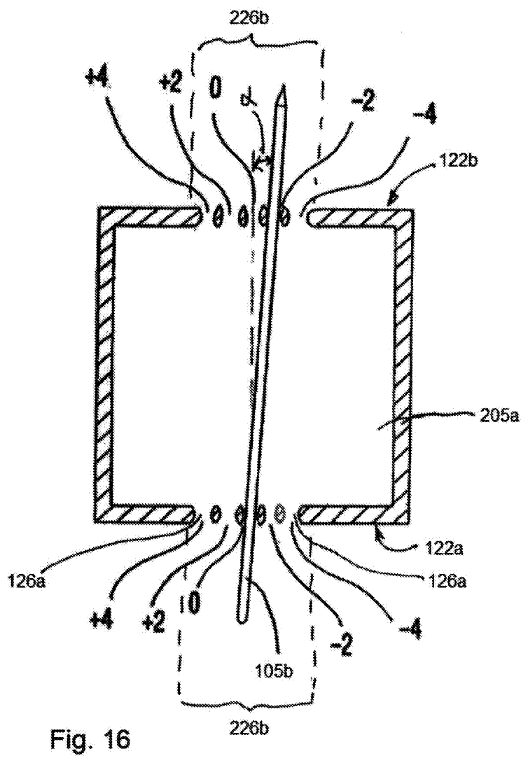

FIG. 16 illustrates a partial view of a cross-section of the multi-perforate cutting block of FIG. 15.

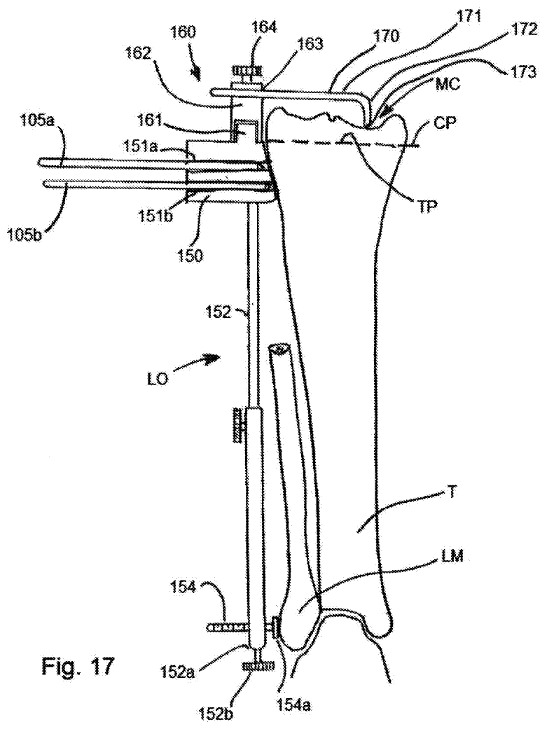

FIG. 17 depicts a locator instrument used in positioning a leveling block relative to the proximal end of a tibia.

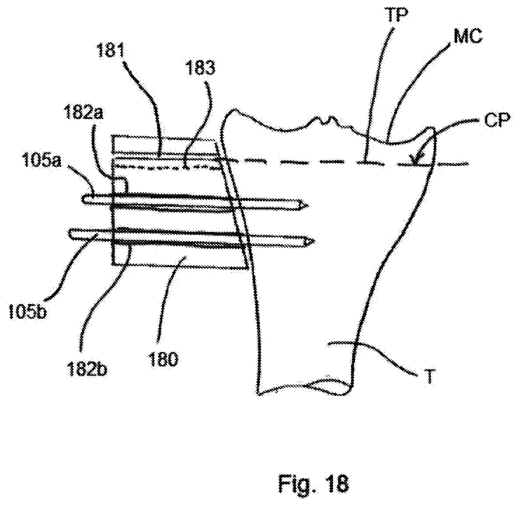

FIG. 18 illustrates a cutting block used for preparing the proximal end of a tibial from a direct lateral approach (lateral direction).

Turning now descriptively to the drawings, in which similar references characters denote similar elements throughout the several views, the attached drawings illustrate my bicondylar knee resurfacing prosthesis (mini bicondylar knee implant) and method for insertion through direct lateral approach and affixation of the femoral component and of the tibial component thereof to surfaces of the corresponding bones using correspondingly shaped retaining clips.

As illustrated in FIG. 1 the femoral component FC comprises two condyles, a medial condyle 1 and a lateral condyle 37 interconnected near a posterior end of each by an intercondylar bridge 38. Each condyle 1, 37 includes having a convex arcuate articular surface 7 and opposite therefrom, a bone contact non-articular surface 6. Preferably the surfaces 6, 7 are polished surfaces, and especially preferably are polished metallic surfaces. This posterior location of the intercondylar bridge is beneficial since it does not interfere with anterior cruciate ligament function.

In the preferred embodiment of the present invention, the bone-contact non-articular surfaces of each condyle 1, 37 comprise a plurality of generally flat adjacent surfaces, respectively for each.

For condyle 1, there is present a posterior bone-contact surface 1a, an anterior bone-contact surface 1c and an intermediate bone contact surface 1b therebetween; and as is more clearly seen from FIG. 2, the angle of posterior bone-contact surface 1a is preferably perpendicular to the angle of the anterior bone-contact surface 1c. Similarly, for condyle 37 there is present a posterior bone-contact surface 37a, an anterior bone-contact surface 37c and an intermediate bone contact surface 37b therebetween; and as is more clearly seen from FIG. 2, the angle of posterior bone-contact surface 37a is preferably perpendicular to the angle of the anterior bone-contact surface 37c. As if further seen, several of these bone-contact surfaces also comprise a recessed receiving channel transverse thereto. In the depicted embodiment a first T shaped receiving channel 5 (also RC1) laterally transverses each of: the posterior bone-contact surface 1a the intercondylar bridge 38 and the posterior bone-contact 37a and thereby defines a first receiving channel 5. As is seen in FIG. 1, the receiving channel 5 has a base art 5b perpendicular to a narrowed neck part 5a defining a gap within the posterior bone-contact surface 1a, the intercondylar bridge 38 and the posterior bone-contact 37a dividing each into plural surfaces. In the depicted embodiment a second T shaped receiving channel (RC2) having two collinear segments 3, 9 is also present and laterally traverses both the anterior bone-contact surface 1c and the anterior bone-contact surface 37c with segment 3 traversing the anterior bone-contact surface 1c and segment 9 traversing the anterior bone-contact surface 37c. It is to be understood that more than two receiving channels may be provided in order to increase the stability of the implanted femoral component FC.

FIG. 4A illustrates a cross-sectional view of the femoral component through reference plane defined by line A and line B (c.f. FIG. 2) illustrating its placement in interfacial contact with resected surfaces of a femur and inserted hourglass shaped retaining clips.

FIG. 4B illustrates a cross-sectional view of the femoral component through reference plane defined by line A and line B (c.f FIG. 2) illustrating its placement in interfacial contact with resected surfaces of a femur and inserted dumbbell shaped retaining clips.

An aspect of the present invention is a novel implant fixation means, namely a shaped retaining clip C which firmly and securely attaches an implant to a resected bony surface. FIG. 5 provide detailed illustration of a shaped retaining clip C. In this depicted preferred embodiment, the retaining clip C has a shaped (or profiled) cross section in the shape of an I beam having a top 15 flange and a bottom flange 16 interconnected with a vertical web 33. Advantageously along the length L of the retaining clip C the top flange 15 diverges from the bottom flange 16 such that the distance B at one end, the distal end 51 is greater than distance A at the opposite, proximal end 50 of the shaped retaining clip C. This establishes that the top flange 15 and bottom flange 16 are non-parallel along their length and angle apart between the proximal end 50 and the distal end 51 by a small amount, namely have a diverging angle of greater than 0° of arc.

Advantageously this diverging angle is greater than 0° but not more than 10°, and more preferably is from about 0.5° and 7.5° and especially preferably is from about 2° and about 7°; such then provides a small taper angle between the top flange 15 and bottom flange 16.

Excessive angle is detrimental since it will cause undesirable bone cavity under the clip. Advantageously, at least one of the flanges, here bottom flange 16 is depicted, and the web 33 have sharp, tapered or beveled end faces 12 and 14 which providing cutting edges 16a and 33a will cut and penetrate inside the bone when impacted with a mallet. While a cutting edge 16a is illustrated on bottom flange 16, it is to be understood that a similar end face and cutting edge may be provided to top flange 15, as is shown with respect to bottom flange 16.

In an alternative to the above, the shaped retaining clip C has a top flange 15 parallel to the bottom flange 16, and a diverging angle of 0°, thus in such an alternative there is no compression provided by the shaped retaining clip C when it is engaged with a component and simultaneously with a bony surface.

It is therefore possible to understand that during the surgical procedure, the top flange 15 of the retaining clip C is introduced in an inverted T slot (receiving channel) of a an implant component, i.e. a femoral component FC (c.f FIG. 1, FIG. 2) which is held against the surface of previously resected bone and then the proximal end 50 impacted with a mallet, urging the shaped retaining clip C into both a receiving channel and the bone in the region of the resected surface and transverse thereto. In view of the divergence of top flange 15 and the bottom flange 16 which is greatest at the distal end 51, the sharp bevel end 12 of the lower flange 16 and its cutting edge 16a will cut and penetrate into the bone at an angle. In doing so, flange 16 will exert a pulling effect on flange 15 via the web 33, which flange 15 will be urged against the bone thereby compressing the implant against the bone. It can thus be realized that any shaped retaining clip C having a taper angle between parts thereof which provided spaced apart, non-parallel surfaces extending along its length L will provide such compressive forces between an implanted component and a bone, and in particular will provide compression between interfacial surfaces of a bone, particularly generally flat or generally planar surfaces of a bone and corresponding surfaces of a prosthetic component (here a femoral component and/or tibial component), which may be used in any part of a body and is not limited to a knee prosthesis but in fact may be used in any joint prosthesis, i.e. shoulder, elbow, ankle, hip.

Optionally but particularly preferably the retaining clip C includes a hole 31 at or near the proximal end 50 of the web 33 whereby an extraction hook (not illustrated) may be introduced to facilitate the positioning of, or the extraction of the retaining clip C during a surgical procedure.

Another optional but preferable additional feature which may be present in a shaped retaining clip C is a locking element LE which preferably includes a resilient part which engages a lock recess 17 present at a suitable position within a receiving channel RC, such that the engagement limits the lateral travel or lateral insertion of the shaped retaining clip C relative thereto. In such a manner over-insertion or excess travel of a shaped retaining clip C relative to an implant component having a suitably correspondingly dimensioned receiving channel RC is hindered. As is seen in FIG. 5, the locking element LE is a resilient thin metallic leaf 29 having a round protrusion 32 constructed from compatible material in a form located within a correspondingly shaped (here, rectangular) recess 30 provided within the surface of one of the flanges 15, 16; here the recess 30 is depicted extending inwardly from the upper surface 15b of the top flange 15. When the retaining clip C is inserted into a corresponding receiving channel (c.f., FIG. 3, RC1, RC2) of an implant component (i.e., femoral component FC), the round protrusion 32 will enter into and engage the lock recess 17 (FIG. 3) thereby locking the retaining clip C at a fixed position, and preventing from further sliding in or out of a receiving channel. Whereas a specific embodiment of a locking element LE has been disclosed and described in the drawings, other parts may be used to ensure a similar function whereby subsequent to proper insertion a retaining clip C is retained or locked at a particular position within a receiving channel RC and/or relative to the component with which the retaining clip C and locking element LE are used. Non-limiting examples of such locking elements include: friction or interference fits between portions of a retaining clip C and a receiving channel RC, implantable wedges forming a friction or interference fits between portions of a retaining clip C and a receiving channel RC, pins, screws, springs, or other ancillary part which halts or hinders the movement of the retaining clip C and a receiving channel RC in which it is present.

Furthermore, the compression clip provides as a guide element a triangular channel 34 (FIG. 5) having a configuration so to receive a guide tool (not shown) having a triangular tip fittable within the triangular channel 34 in order to help manipulate and hold the clip during insertion. The triangular channel 34 itself may be threaded, and/or may be substituted by a different configuration, i.e. a bore or hole which may be threaded, with such threads being of a configuration to engage mating threads on a removeable guide tool (not shown). In addition, a circular hole 31 near the outer end of the vertical web 33 used to insert a hook (not shown) in order to remove and pull out the compression clip.

The materials of construction used to form the various component parts of the bicondylar knee resurfacing prosthesis disclosed herein are necessarily biocompatible, and preferably include those known to the present art. Such include metals, polymers and ceramic materials. Preferably at least one of the interfacial contact surfaces between the femoral component and the tibial component is a polymer which provides a smooth wear surface within the prosthetic joint. Advantageously at least the condyles of the femoral component FC are surfaced with a hard coating, such as a metal, metallic or ceramic material but most advantageously the condyles 1, 37 are formed of a metal or metallic material of construction. Concurrently the surface coming into direct contact with the condyles 1, 37 is a softer material, which again may be a metal, metallic or ceramic material but most advantageously is a polymeric material such as polyethylene or other suitable synthetic polymer. The materials of construction of the retaining clip C is also made of a suitable biocompatible material and preferably is fabricated from or fabricated using materials compatible with those used in the other component parts. In one embodiment the retaining clip C is made of or using a compatible metal or metallic alloy (i.e., titanium or cobalt chrome alloy) having the same or similar composition as the implant component (i.e, femoral component, tibial component) with which it is used in order to avoid any galvanic corrosion caused by contact between dissimilar metals. In a further embodiment the retaining clip C is made of or using resorbable materials such as biodegradable polymers PLLA and other and resorbable composites commonly used in orthopedics. Notwithstanding the fact that the disposable retaining clips can be made from compatible and similar metal to the component they are inserted into, such as titanium or cobalt chrome in order to avoid bi-metal galvanic corrosion.

Turning now to FIG. 6, visible in this cross-sectional view is an embodiment of a tibial component TC which includes a tibial tray 19, which implanted upon a resected and prepared proximal tibial end E of a tibia T. The tibial tray 19 has a flat upper surface 34 and a peripheral rim 21 at its margins so to contain the polyethylene insert part 20. In the view of FIG. 6, while the top face 20a of the insert part 20 is depicted as being flat, it may have a different non-flat surface profile; in a preferred embodiment two correspondingly configured recesses or cupules CC corresponding to the condyles 1, 37 extend inwardly from the top face 20a. The tibial tray 19 is firmly attached to the tibial end E via two or more I-beam shaped retaining clips 26 and 27, each having a part inserted through corresponding T shaped receiving channels 25 and 28; as to be understood in conjunction with the foregoing remarks, the base surface 19b of the tibial tray 19 is compressed against and is in interfacial contact with the tibial end E. The insert part 20 is at least partially fitted within the inner sidewall 21a of the peripheral rim 21 such that its base surface 20b is in register with the top surface 19a of the tibial tray 19. Such may be facilitated by providing a recessed margin 20c adjacent to base surface 20b of the insert part 20. The insert part 20 may be firmly retained in its position via a linking clip 22 which is insertable into a linking channel 41 which extends through and is in part beneath the top surface 19a and includes an inlet 40a passing inwardly from the outer sidewall 21b of the peripheral rim 21, and terminates at an end stop 40b, which does not extend completely across the top surface 19a of the tibial tray 19. The cross-sectional geometry of the linking channel 41 may be of a similar configuration as a receiving channel, and the corresponding cross-section of a linking clip 22 may be of a similar configuration as a shaped clip SC, (here, the linking clip 22 has an I-beam shaped cross-section, as does securing clip 26 and further securing clip 27) but the linking clip 22 need not necessarily have to have a small taper angle between the its top flange 22a and its bottom flange 22b, but its top flange 22a and its bottom flange 22b may be parallel, thus lacking provision of a compressive effect between the insert part 20 and the tibial tray 19 part when the linking clip 22 is inserted in linking groove 41 of the tibial tray 19 (47) and in corresponding linking groove 41a provided in the insert part 20. As previously noted, advantageously two concave cupules, generally longitudinal trenches in shape, oriented from the front to the back are also present in the insert part 20 and will receive the two condyles 1, 37 of the implanted femoral component FC.

In a different embodiment of the present invention illustrated in FIG. 7, the tibial tray 19 of the tibial component TC is designed to spare the ACL (anterior cruciate ligament), which is usually sacrificed during the preparation of the tibial plateau. A large anterior indentation 35 is provided that allow space for the ACL and avoid interference during the entire arc of motion of the femoral component. This is more clearly seen in FIG. 7A, extending inwardly from the top face 20a of the insert part are two correspondingly configured recesses or cupules CC corresponding to the condyles 1, 37.

In certain situations the surgeon may be faced with no alternative but to sacrifice the ACL, or that the ACL is undesirably loose, or slack, or partially torn and in need of repair or replacement. In such a situation the surgeon may elect to sacrifice the ACL and replace it with a graft. Such a graft may be implanted according to common arthroscopic ACL replacement, via tibial and femoral tunnels. In such a situation, the graft passes through the large anterior indentation 35 of the tibial tray 19, as illustrated FIG. 7, whose large dimensions easily accommodate the graft. This method allows the surgeon to restore the mobility and kinematics of the knee.

FIG. 8 is a perspective view of further embodiment of a bicondylar tibial tray 19, also illustrating receiving channels 25, 28 (T shaped receiving channels) configured to receive correspondingly shaped retaining clips which affix the tibial tray 19 in interfacial contact with a resected surface of a tibia T; also seen is a T shaped linking channel 41 used in retaining the insert 19 component, preferably formed of polyethylene. A view of the tibial tray 19 of FIG. 8 implanted upon the end of a tibial T is shown on FIG. 8A, wherein is also seen extending inwardly from the top face 20a of the insert part are two correspondingly configured recesses or cupules CC corresponding to the condyles 1, 37.

It is to be noted that in particularly preferred embodiments, the femoral component FC does not address the femoro-patellar articulation and the mini knee implant components make no contact with the patella during the entire range of motion.

Regarding the initial period of implant fixation compression provided by the above described clips is advantageous since fixation of the implant primarily depends on bone ingrowth into the asperities of the bone contact surface. However, this integration would take as much as 4 to 6 weeks to develop, during that phase, the retaining clip provides excellent initial fixation.

A method of implanting the bicondylar knee resurfacing prosthesis and parts thereof are now discussed, as well as specific instrumentation used with one or more parts of the bicondylar knee resurfacing prosthesis. The bicondylar knee resurfacing prosthesis and parts thereof discussed herein provide for a method of inserting the mini bicondylar implant through a small surgical incision made over the lateral aspect of the operated knee away from the patella and without any disruption or damage to the quadriceps muscle or the patellar tendon. With reference now to FIG. 9, as opposed to conventional surgical total knee replacement surgery, where the patient lies supine on his back, the surgical technique utilized in the present invention is performed with patient lying on his side on the operative table. The operated knee is positioned upon a surgical platform 95 firmly attached to the side rails 96 of the operating table 90 with adjustable brackets 91; here four such brackets are shown, but a lesser or greater number may also be used. Parts of the leg are then secured to the platform 95 using strong commercial grade wide Velcro 94 that will firmly but removably affix a stockinet 93 wrapped around the lower leg and holds the leg firmly attached to the platform 95 during most of the surgical implantation procedure.

With reference now to FIG. 10, in order to perform the bone resection at the end of the femur F, the operating surgeon must delineate the three resection planes, which will remove the weight bearing articular surface of the medial femoral condyle MC, and the lateral femoral condyle LC. This process first requires the use of a hand-held locator apparatus. A hand held femoral template 100 is used, an embodiment of which is shown. The femoral template 100 includes a grippable handle 29 which is used to position the femoral template 100 relative to part of the femur exposed through a lateral surgical incision in the locus of the knee joint. Extending from the handle 101 is an outrigger 102 and a bracket 102a having two sleeves 103 which accommodate and orient two stabilizer rods 105a, 105b over the lateral aspect of the femur at the proximal end of the incision, without the need to provide a further incision. The hand held femoral template comprises 100 two articular arcuate pads 107a, 107b connected together by adjustable connecting rod 108, the said pads 107a, 107b are placed over the corresponding articular surfaces of the lateral condyle and the medial condyle and manually held in place by handle 101, and next are firmly secured in place with two small pins introduced through holes 27. Thereafter, the two stabilizer rods 105a, 105b are removably affixed to the exposed surface of the lateral condyle LC, i.e., by drilling or other means, and subsequently the pins in holes 27 are removed, and the femoral template 100 withdrawn, leaving behind the two parallel, spaced apart stabilizer rods 105a, 105b upon which may be mounted other instruments, i.e, cutting blocks used in resection of the bone. Such cutting blocks function as templates for subsequent resection of bony condyle surfaces and for later insertion of retaining clips RC with femoral components FC of the bicondylar knee resurfacing prosthesis.

Turning now to FIG. 11, an embodiment of a cutting block 120 used to delineate the position of resected surfaces and of retaining clips is illustrated. As is visible from the figure, the cutting block 120 is a three-dimensional body having a thickness 121 between two parallel spaced apart faces, here 122a is visible, and while not visible a corresponding face 122b is not visible, it is to be understood to be symmetrical with that of face 122a. The cutting block 120 is slidingly mounted upon the two parallel, spaced apart stabilizer rod 105a, 105b via corresponding guide holes 126a, 126b and is movable relative thereto. Face 122b is advantageously near, or is in physical contact with the lateral condyle LC. The cutting block 120 also comprises cutting slots, here a horizontal cutting slot 128a, a chamfer cutting slot 128b and a vertical cutting slot 128c which are used to delimit the direction of motion of a conventional surgical cutting instrument, especially preferably a reciprocating surgical saw which has a blade insertable within each of these cutting slots which is used to resected surfaces of the lateral condyle LC and the medial condyle MC; such resection is performed in order to provide generally flat, posterior bone-contact surface 37a, anterior bone-contact surface 37c and intermediate bone contact surface 37b (c.f. FIG. 2). As is visible in FIG. 11, the cutting block 120 also includes correspondingly configured guide channels 129a, 129b which is used to position the location of the penetrating osteotome PO (c.f. FIG. 12). The penetrating osteome PO is generally rod-like in shape and has a specifically profiled cutter section 130 having a cutting end 131, and at grippable part 136 which terminates at a striking end 137, which here includes a larger striking head 137a which is used to receive the blows a mallet, or to receive a compressive force. The lateral cross-section (as may be defined along line x-x) of the cutter section 130 and of the cutting end 131 is dimensioned to be received within one of the correspondingly configured guide channels 129a, 129b. Here, the lateral cross-section of the cutter section 130 is of an I-beam shape, and includes a top plate 132a and a bottom plate 132b parallel to the top plate 132a, interconnected by an intermediate, perpendicular web 132c, here the size of the top plate 132a and the bottom plate 132b are essentially of the same size, and have the same width, as well as length. At the cutting end 131 the bottom plate 132b and the web 132c have sharp, tapered or beveled end faces 131b, 131c while the end face 131a of top plate 132a does not. The sharp, tapered or beveled end faces 131b, 131c providing cutting edges which will cut and penetrate inside the bone when striking end 137 is impacted, i.e, with a mallet. The penetrating osteoma PO is first inserted into a guide channel, i.e. guide channels 129a, 129b (here guide channel 129a is depicted) such that the top plate 132a is located within the cutting slot 128a, the whereby the web 132c and bottom plate 132b are received within and slideable through the cutting block 120; the length of the cutter section 130 is sufficiently long such that the cutting end 131 can be driven substantially into, or through, the femur adjacent to a resected surface thereof. The use of the penetrating osteotome PO prepares the femur for subsequent insertion of a retaining clip RC.

Further cutting blocks 120 are depicted on FIG. 13A and FIG. 13B, illustrating alternative embodiments. In FIG. 13A are depicted guide channels 129a, 129b which are used with retaining clips RC (not shown here) having a dumbbell cross-sectional profile. In FIG. 13B are depicted guide channels 129a, 129b which are used with retaining clips RC (not shown here) having a hourglass shaped cross-sectional profile.

FIG. 14 depicts a cutting block 120 removably mounted upon stabilizer pins 105a, 105b over the lateral aspect of the distal femur F at the proximal end of the surgical incision SI illustrating its lateral location relative to the knee joint. Through a true lateral approach, the surgeon exposes the lateral surface of the knee. In flexion, the lateral collateral ligament is retracted posteriorly to enhance the exposure.

In some patients, the damage to the articular cartilage from trauma or arthritis may cause misalignment of the articular condyles. In other words, the condyles may not have the same height and variation of the previously described cutting block 120 is provided, here a multi-perforate cutting block 200 as illustrated on FIG. 15 and FIG. 16. The cutting block 200 is similar in most respects with the cutting the block 120 discussed previously, in particular with reference to FIG. 11, FIG. 13A, FIG. 13B and FIG. 14 but differs in that instead of a single pair of guide holes 126a, 126b a plurality of guide holes, or a guide hole array 226a, 226b is provided. As is seen from FIG. 14, each guide hole array 226a, 226b includes a patterned arrangement of individual guide holes which are in a regular pattern and within each pattern, or at a particular position and/or relative distance from a next or adjacent guide hole within the same guide hole array 226a, 226b. In the embodiment of FIG. 14 and FIG. 15 are illustrated guide hole arrays 226a, 226b which each have identical patterns of individual guide holes (and in each, the guide holes are delineated by one of the labels A, B, O (for the center most guide hole), +4, +2, −2 and −4) which perforate face 122a; while here, face 122a is visible and parallel corresponding face 122b is not visible in this figure, it is to be understood to be symmetrical with that of face 122a. Preferably the space between opposite guide hole arrays 226a, 226b are hollowed cavities 205a, 205b. As opposed to the provision of only two guide holes 126a, 126b, provision of guide holes arrays 226a, 226b on opposite faces 122a, 122b (c.f FIG. 15) of a cutting block 220 allows for some degree of angular offset of the cutting block 220 as opposed to the cutting block 120 having only two parallel guide holes 126a, 126b. As is best understood with reference to FIG. 15, (a cross-section of the cutting guide 200 of FIG. 14 at a reference plane defined by reference line labeled ‘Y-X’ on FIG. 15) the guide hole arrays 226b allow for the insertion of a stabilizer pin 105a, through individual holes on each of the guide holes arrays 226a, 226b on opposite faces 122a, 122b wherein the stabilizer pin 105a passes through different holes in each of the guide hole arrays 226b on opposite faces 122a, 122b. This is clearly seen on FIG. 15 wherein stabilizer pin 105a passes through the guide hole “0” of the guide hole array 226b on face 122a, but passes through a different hole, “−2” of the symmetrically configured guide hole array 226b on opposite, parallel face 122b, which introduces an offset angle “α” relative to a perpendicular axis which is defined as passing through both of guide holes “0” of each guide hole array 226b on both face 122a, 122b. This variability allows for the variable and offset placement of the perforate cutting block 220 relative to the femur F, and varies the cutting angle of any saw or cutter used with the horizontal cutting slot 128a, a chamfer cutting slot 128b and a vertical cutting slot 128c which are used to delimit the direction of motion of a conventional surgical cutting instrument resecting the bone.

FIG. 17 depicts a locator instrument LO used in positioning a leveling block relative to the proximal end of a tibia T. As it appreciated from the drawing figure, the locator instrument LO makes possible the determination of a cutting plane CP at the proximal end of the tibia which forms a tibial plateau TP upon which a tibial tray 19 is affixed. The position of the cutting plane is offset from the lowest point of a condyle, usually the medial condyle MC and this offset is usually about 2-10 mm, more preferably about 2-5 mm below the lowest point on the interior of the condyle. This may be established by the locator instrument LO which includes a leveling block 150 having two (or more) bores 151a, 151b passing therethrough which are sized to accommodate and orient two (or more) stabilizer pins 105a, 105b over the adjacent proximal end of the tibia T. Depending from a part of the leveling block 150 of the locator instrument LO is a placement rod 152, here shown as a rod having sliding telescoping section which at or near a distal end 152a thereof is an offset rod 154 which transversely passes (or which can be affixed) through the placement rod 152 and whose position can be varied and temporarily affixed, such as by use of a threaded locking knob 152b. The offset rod 154 has an end 154a which is positioned against the lateral malleolus LM of the ankle joint and the position of the distal end 152a relative to the end 154a can be varied until a suitable positioning can established by a surgeon, and thereafter the offset rod 154 may be temporarily locked by turning the locking knob 152b. This establishes the relative position of the leveling block 150 to the ankle, as well as the relative position, including an angular position or angular offset of the leveling block 150 relative to the tibia. Depending from a further part of the leveling block 150 is a stylet holder 160 which in the current embodiment includes a base part 161, upon which is positioned a moveable, i.e. rotatable turret part 162 which may rotated relative to the leveling block, and preferably is either collinear or has a central axis of rotation which is parallel to the central axis of the placement rod 152. The stylet holder 160 also includes a stylet mount 163 which grips a long arm 171 of a stylet 170, and in the embodiment a stylet holder lock nut 164 is provided which may be rotated to release or grip a part of a stylet 170. With reference now to the depicted stylet 170, such is generally rod shaped and has a long arm 171 and a short arm 172 which terminates at a stylet tip 173. The stylet tip 173 is used with the stylet holder 160 to locate the lowest point on the interior of the condyle in which it comes into contact. As the length from the stylet tip 173 to the mid-line of the long arm 171, and the dimensions of all other parts of the locator instrument LO are known or can be determined, the relative position of the leveling block 150 to the stylet tip 173, and the position of the two (or more) bores 151a, 151b passing therethrough relative to the tibia T can be established. It is to be understood that the locator instrument LO may be used with stylets 170 of varying dimensions and sizes including ones having short arms 172 of different lengths, and thus different lengths from the stylet tip 173 to the mid-line of the long arm 171; careful selection of a stylet 170 can thus be used to establish the location of a cutting plane CP at the proximal end of the tibia which forms a tibial plateau TP upon which a tibial tray 19 is to be affixed. Once the relative position of the leveling block 150 relative to the tibia T is determined by using both the stylet 160 and the placement rod 152, the two (or more) stabilizer pins 105a, 105b can be driven into a part of the adjacent proximal end of the tibia T. Subsequently, the locator instrument LO may be removed.

FIG. 18 illustrates a tibia cutting block 180 used for preparing the proximal end of a tibial T for resection in order to form a tibial plateau TP. The tibia cutting block 180 includes a guide slot 181 for receiving a cutting instrument and two (or more) of the two (or more) bores 182a, 182b; the position of these bores correspond to the position of the two (or more) bores 151a, 151b of the leveling block 150. The tibia cutting block 180 may be slidably mounted on the two (or more) stabilizer pins 105a, 105b can be driven into a part of the adjacent proximal end of the tibia T. The relative position of the guide slot 181 can be established to be coincident with the cutting plane CP, and the cutting instrument, preferably a reciprocating surgical saw is inserted through the guide slot 181 and is guided thereby, thus providing controlled resection of the end of the tibia T and the formation of the tibial plateau TP, suited to receive a tibial component TC. Although not fully visible in the cross-sectional view of FIG. 18 it is to be understood that the tibia cutting block 180 also includes one or more correspondingly configured guide channels (c.f, guide channels 129a, 129b of FIG. 14) which is used to position the location of the penetrating osteotome PO (c.f FIG. 12), which is used to prepare the tibia T for receiving a tibial component TC and insertable shaped retaining clip(s) C which are slidably inserted through the same lateral approach. In FIG. 18, a part of a guide channel 183 is illustrated by a broken line adjacent to the guide slot 181; reference here is made to FIG. 12 which explains the relative positioning and use of the penetrating osteoma PO relative to the guide channel(s) 183 present in the tibia cutting block 180.

It is to be appreciated that the use of the locator instrument LO and the tibia cutting block 180 permits for the preparation of a tibia T for subsequent implantation of the tibial component TC from a direct lateral approach (lateral direction), and this does not require that the kneecap be removed or ‘flipped’, and also allows for the implantation of a bicondylar knee resurfacing prosthesis without causing damage of the Anterior Cruciate Ligament during the lateral insertion of the femoral and tibial components. This will preserve the physiological function of the knee and allow range of motion to be kinematically as close as possible to normal knee.

It is to be understood that according to the surgical technique the femur may be prepared i.e., resected, prior to the preparation, i.e., resection of the tibia, or vice-versa. (‘mutatis mutandis’) In some embodiments, initial resection of the tibia and its preparation to receive a tibial component TC provides for increased access to the internal cavity of the knee, thus allowing more space for the preparation of the femur for receiving the femoral component FC.

It will be understood that various modifications may be made to the embodiments disclosed herein. Therefore, the above description should not be construed as limiting, but merely as exemplifications of embodiments. Those skilled in art will envision other modifications within the scope and spirit of the claims appended hereto.

1. A bicondylar implantable knee prosthesis for resurfacing the weight bearing surface of the femur and tibia comprising:
a convexly curved medial condyle having a curved outer articular surface and lateral condyle having a curved outer articular surface, and an intercondylar bridge; and
a concave inner surface that has at least one receiving channel, wherein, when the prosthesis is implanted on the weight bearing portion of the femur, the entire outer articular curved surface does not resurface the femoro-patellar joint and wherein, the prosthesis is configured to be inserted through direct lateral approach, and retaining clips to be inserted laterally into the at least one receiving channel.

2. The prosthesis of claim 1, wherein at least receiving channel transversally crosses the nonarticular inner surface of the medial and lateral condyles.

3. The prosthesis of claim 2, wherein the inner surface has a plurality of receiving channels.

4. The prosthesis of claim 1, wherein the inner surface has a receiving channel within the intercondylar bridge.

5. The prosthesis of claim 1, wherein the retaining clip has a cross sectional design of an I beam comprising a top and a bottom parallel flanges connected by a vertical web whereby one flange of the I beam will be inserted slidingly into the receiving channel and the other flange having a sharp edge will penetrate into bone when the clip is impacted with a mallet.

6. The prosthesis of claim 1 wherein the retaining clip has diverging flanges wherein when impacted into bone, the retention compresses the implant against adjacent bone.

7. The prosthesis of claim 1, wherein the retaining clip is constructed from compatible metallic alloy, plastics or resorbable material.

8. A bicondylar implantable knee prosthesis for insertion between femur and tibia comprising:
a medial and lateral curved articular femoral condylar components and an intercondylar bridge, an inner nonarticular surface including:
at least one receiving channel,
a tibial platform having a bottom surface, which includes at least one tibial transverse receiving channel and a top surface which includes a circumferential retaining rim, said tibial platform provides a receiving channel for retaining the polyethylene insert,
a polyethylene insert having a top surface, which provides two condylar depressions or grooves to articulate with the condylar articular surface of the femoral component and a receiving channel for retaining the polyethylene insert,
wherein the femoral component is implantable through lateral insertion through a direct lateral approach to the retropatellar region, and,
wherein the tibial platform is implantable through the same lateral incision, wherein the polyethylene insert is implantable through the same lateral incision.

9. The prosthesis of claim 8, wherein the femoral bicondylar component is secured to the femoral cut surface by at least retaining clip which both affixes and compresses the femoral component against adjacent bone.

10. The prosthesis of claim 8, wherein more than one retaining clip is used to firmly secure the implant against bone.

11. The prosthesis of claim 8, wherein the tibial component is secured to the tibial cut surface by at least one retaining clip to affix and compress the metallic tibial component against bone.

12. The prosthesis of claim 8, wherein the polyethylene insert is secured to the tibial platform using at least one retaining clip.

13. A method of resurfacing the weight bearing surface of the femur and tibia comprising the step of:
inserting the prosthesis of claim 1 through a small surgical incision made over the lateral aspect of the operated knee away from the patella and without any disruption or damage to the quadriceps muscle or the patellar tendon.

14. A method of resurfacing the weight bearing surface of the femur and tibia comprising the step of:
inserting the prosthesis of claim 8 through a small surgical incision made over the lateral aspect of the operated knee away from the patella and without any disruption or damage to the quadriceps muscle or the patellar tendon.
